(19) United States
(12) Patent Application Publication
(10) Pub. No.: US 2023/0293345 A1
(43) Pub. Date: Sep. 21, 2023

(54) IMPROVEMENTS IN OR RELATING TO A VISUAL GUIDE

(71) Applicant: Dispenser Technologies Limited, Waterlooville Hampshire (GB)

(72) Inventors: Raymond John BACON, Waterlooville Hampshire (GB); Benjamin Peter HALL, Waterlooville Hampshire (GB); Martin Christopher BUNCE, Waterlooville Hampshire (GB)

(73) Assignee: Dispenser Technologies Limited, Waterlooville Hampshire (GB)

(21) Appl. No.: 18/017,200

(22) PCT Filed: Jul. 21, 2021
(86) PCT No.: PCT/GB2021/051869

§ 371 Date: Jan. 20, 2023

(30) Foreign Application Priority Data
Jul. 22, 2020 (GB) 2011323.9

(51) Int. Cl. A61F 9/00; A61B 90/00

(52) U.S. Cl. CPC A61F 9/0026; A61B 90/361; A61B 2090/0807

(57) ABSTRACT

A visual guide for assisting a user with the positioning of an object relative to the user’s eye is provided. The visual guide comprises: a first positioning indicator provided on an axis; and a second positioning indicator, visually distinct from the first. The first and second positioning indicators are offset along the axis. Furthermore, visual contact of the first and second positioning indicators occurs when the object is located within a predetermined locus of the user’s eye.

This invention relates to improvements in or relating to a visual guide, and in particular, to a visual guide for assisting a user with the positioning of an object relative to their eye.

Accurately positioning an object with respect to your own face and/or eye can present many challenges. One such challenge results from the fact that as an object moves closer to the eye, it becomes increasingly difficult to focus on the object and your visual perspective can become misleading. It can therefore become difficult to tell whether the object is at an appropriate distance from the face/eye and/or whether the object is correctly aligned with the face/eye. These challenges are seen in a variety of different sectors, including the security, medical and recreational sectors.

For example, modern technology has resulted in devices, such as cameras, scanners and detectors, which can accurately and reliably image and/or scan the human body. As this technology continues to develop the amount of data obtainable from a single image or scan increases, which enables more localised and/or more accurate data to be obtained. Moreover, the continuous development of such technology is resulting in its affordability within more common everyday situations, such as face and/or eye recognition passwords, personal identification and for the determination of certain illnesses and their symptoms.

Accordingly, the accuracy with which an imaging or scanning device is positioned relative to the human body is becoming increasingly important. Furthermore, the ease and speed with which an appropriate positional relationship between the device and the human body is found are also becoming increasingly important. For sensitive medical and security devices, users are often required to place their forehead, chin and/or another body part in contact with a positioning guide. Such positioning guides are often uncomfortable and require thorough cleaning between users. In some applications, the time taken to clean these positioning guides is impractical.

In addition to obtaining the correct position of an imaging or scanning device relative to a user’s body, there is also the need to position medicament dispensers and medical devices relative to a patient’s body and, with a specific reference to ophthalmic medicaments, relative to a patient’s eye. A visual guide for enabling the accurate positioning of an object relative to the human body within a suitable time frame is therefore also sought.

It is against this background that the present invention has arisen.

According to the present invention, there is provided a visual guide for assisting a user with the positioning of an object relative to the user’s eye, the visual guide comprising: a first positioning indicator provided on an axis; and a second positioning indicator, visually distinct from the first, wherein the first and second positioning indicators are offset along the axis and wherein visual contact of the first and second positioning indicators occurs when the object is located within a predetermined locus of the user’s eye.

The present invention provides a low cost, simple and easy to manufacture visual guide that can be used for a variety of different applications. The guide enables users to position an object, such as an imaging device or ophthalmic fluid dispenser, at a suitable distance from and in correct alignment with their eye.

Moreover, the claimed visual guide is intended to be ‘contact-free’ and thus users do not necessarily have to touch any of the components in order for the invention to work, although this may vary depending on the application in which the visual guide is being used. For example, the user may move with respect to the visual guide and/or may move the visual guide with respect to themselves. Alternatively, or in addition, the user may move their head, face or eye with respect to the visual guide and/or may move the visual guide with respect to their head, face or eye.

The visual guide is intended to provide feedback to the user to enable them to position an object at a suitable distance from their eye and to suitably align the object with their eye. Suitable alignment with the user’s eye may be determined with respect to the visual axis of the user’s eye, wherein the visual axis of the user’s eye is defined by a straight line that passes through both the centre of the pupil and the centre of the macula. The centre of the macula is commonly known as the fovea, central fovea or fovea centralis. These two requirements define a locus in which the user should aim to position the object. There is an ideal position within these suitable positions, in which the first and second positioning indicators are precisely aligned. Around this optimum position, there is a penumbra of positions that, whilst not perfect, may be fit for function. This penumbra of positions is included within the locus of predetermined positions which are deemed acceptable. Within this penumbra of positions are positions where at least a first portion of the first positioning indicator appears to touch at least a first portion of the second positioning indicator, thus resulting in visual contact of the first and second positioning indicators. Positioning indicators that appear to touch in three-dimensional (3D) space would physically touch if they were projected onto a two-dimensional (2D) plane perpendicular to the visual axis.

Accordingly, the visual guide for assisting a user with the positioning of an object relative to the user’s eye may comprise: a first positioning indicator provided on an axis; and a second positioning indicator, visually distinct from the first, wherein the first and second positioning indicators are offset along the axis and wherein visual contact of the first and second positioning indicators occurs when the object is located within a predetermined range of distances from the user’s eye and when the object is positioned within a predetermined range of angles relative to the visual axis of the user’s eye.

For example, the object may be configured to dispense a fluid. The visual guide may be configured such that visual contact of the first and second positioning indicator occurs when the object is positioned to dispense the fluid into the user’s eye. More specifically, the object may be an eye drop dispenser.

Alternatively, the object may be a camera, for example. The visual guide may be configured such that visual contact of the first and second positioning indicator occurs when the camera is positioned directly in front of and at a predetermined distance from the user’s eye and/or face.

At least one of the first and second positioning indicators may comprise at least one point, line or area. In some embodiments, at least one of the first and second positioning indicators may comprise a plurality of points, lines and/or areas. Furthermore, in some embodiments, at least one of the first and second positioning indicators may be a single point, line or area.

It should also be appreciated that the position of the object may be moved relative to the eye; the position of the eye may also be moved relative to the object or both the object and the eye may be moved relative to each other, simultaneously.

Visual contact of substantially all of the first indicator with at least a portion of the second positioning indicator may occur when the object is located within a predetermined range of distances from the eye and when the object is positioned within a predetermined range of angles relative to the visual axis of the user’s eye.

The predetermined range of distances from the eye may be within the predetermined locus of the user’s eye. The predetermined range of angles relative to the visual axis of the user’s eye may be within the predetermined locus of the user’s eye.

Visual contact of substantially all of the first positioning indicator with at least a portion of the second positioning indicator ensures that the object is both within a predetermined range of distances from the eye and is within a predetermined range of angles relative to the visual axis of the user’s eye. The predetermined range of distances from the eye may vary depending on the object. For example, the predetermined range of distances from the user’s eye may be less than 1 cm, 5 cm, 10 cm, 20 cm, 30 cm, 50 cm, 75 cm, 100 cm, 150 cm, 200 cm or more than 200 cm. Moreover, the predetermined range may also start at a distance of less than 1 cm, 5 cm, 10 cm, 20 cm, 30 cm, 50 cm, 75 cm, 100 cm, 150 cm, 200 cm or more than 200 cm from the lens of the user’s eye.

The predetermined range of angles relative to the visual axis of the user’s eye may vary depending on the object. For example, the predetermined range of angles relative to the visual axis of the user’s eye may be less than 1 degree, 5 degrees, 10 degrees, 20 degrees, 30 degrees, 40 degrees, 50 degrees, 60 degrees, 70 degrees, 80 degrees, or 90 degrees. Moreover, the predetermined range may also start at an angle of less than 1 degree, 5 degrees, 10 degrees, 20 degrees, 30 degrees, 40 degrees, 50 degrees, 60 degrees, 70 degrees, 80 degrees, or 90 degrees relative to the visual axis of the user’s eye. The predetermined range of angles may be measured between a longitudinal axis of the object and the visual axis of the user’s eye. Alternatively, the predetermined range of angles may be measured between at least a portion of the object and the visual axis of the user’s eye.

The first and second positioning indicators may be coaxial. Coaxial first and second positioning indicators may be used to ensure that a preselected axis of the object, such as its longitudinal axis, is aligned with the visual axis of the user’s eye. Conversely, non-axial alignment of the first and second positioning indicators may enable a user to position an object within a predetermined range of angles, or at a specific angle, relative to the visual axis of the user’s eye. The angle may be an acute angle. Non-coaxial first and second positioning indicators may be asymmetric about at least one axis. For example, the positioning indicators may be oval-shaped and may be configured to appear concentric when viewed from a predetermined angle.

At least one of the length, circumference, diameter and cross-sectional area of the first positioning indicator may be less than the corresponding measurement of the second positioning indicator. Alternatively, or in addition, the first positioning indicator may fit substantially within an external boundary of the second positioning indicator when viewed in two-dimensions (2D). This may result in a first positioning indicator which is smaller, when considered as a whole, than the second positioning indicator. A smaller first positioning indicator enables a user to see both positioning indicators simultaneously when the visual guide is a certain distance from the eye. When the guide is moved towards the eye, the first positioning indicator may begin to obscure the second positioning indicator. At an optimal distance from the eye, the first positioning indicator may completely obscure the second positioning indicator.

The first positioning indicator may be a different and/or contrasting colour to the second positioning indicator. A different and/or contrasting colour enables a user to more accurately determine when the first and second positioning indicators are in visual contact or alignment even when the guide is too close to the user for their vision to optimally focus. For example, the first positioning indicator may be red and the second positioning indicator may be blue. However, it will be appreciated that any number of different and/or contracting colours may be chosen, including as black and white, for example, which may be more suitable for those who are colour blind. Moreover, any number of different shades and varieties of the same colour may also be used, such as light blue and dark blue.

The first positioning indicator may comprise a different and/or contrasting colour, pattern and/or luminescence to the second positioning indicator. For example, the first positioning indicator may be a first pattern, such as a solid colour whereas the second positioning indicator may be a second pattern, such combination of striped colours or different coloured dots/shapes. Again, it will be appreciated that any combination and different arrangement of patterns and colours may be used on either the first and/or second indicator.

At least one of the first and second positioning indicators may be configured for attachment to the object. Attaching the first and/or second positioning indicators to an object allows the guide to be used with existing objects or devices without any need to adapt the object or device.

The first positioning indicator may comprise a plurality of discrete elements. Alternatively, or in addition, the second positioning indicator may comprise a plurality of discrete elements. A plurality of discrete elements enables a positioning indicator to include a plurality of discrete areas, lines or even points, such as each tip of a tringle or each corner of a square, for example. This enables very precise positioning of the object with very little tolerance, thus enabling the object to be positioned more accurately. For example, if the guide is too close to the eye, the discrete elements of the first positioning indicator may be visually outside of the second positioning indicator or may entirely obscure the second positioning indicator. Conversely, when the guide is too far from the eye, the discrete elements of the first positioning indicator may be entirely within the second positioning indicator or may not yet visually touch a portion of the second positioning indicator.

The plurality of discrete elements of the first positioning indicators may comprise a different and/or contrasting colour, pattern and/or luminescence to the plurality of discrete elements of the second positioning indicator.

At least one of the length, circumference, diameter and cross-sectional area of at least one element of the first positioning indicator may be less than the corresponding measurement of the second positioning indicator. Alternatively, or in addition, at least one element of the first positioning indicator may fit substantially within an external boundary of at least one element of the second positioning indicator when viewed in two-dimensions (2D). This may result in an element of the first positioning indicator which is smaller when considered as a whole than the equivalent element of the second positioning indicator. A first positioning indicator enables a user to see elements of both positioning indicators, simultaneously, when the visual guide is beyond a certain distance from the eye the predetermined locus of the user’s eye. When the guide is moved towards the eye, at least one element of the first positioning indicator may come into visual contact and/or begin to obscure at least one element of the second positioning indicator. At an optimal distance from the eye, at least one element of first positioning indicator may completely obscure at least one element of the second positioning indicator.

At least one positioning indicator, or at least one element thereof, may be at least one of:
i. An outer surface or boundary of a ring or disc;
ii. A continuous line or marking on a ring or disc;
iii. A plurality of discrete markings on a ring or disc;
iv. An outer surface of at least three protrusions or outcrops;
v. A continuous marking or scale on at least three protrusions or outcrops.

At least one of the first and second positioning indicators may be integral to the object. Integrating the visual guide into or onto the object reduces the number of components required and thus reduces material. It also ensures that the object and guide to remaining together and therefore prevents the guide from being moved, incorrectly positioned relative to the object or from being lost altogether. At least one of the first and second positioning indicators may be permanently attached to the object. Alternatively, or in addition, at least one of the first and second positioning indicators may be a component of the object.

At least one of the first and second positioning indicators, or at least one element thereof, may be at least one of.
i. A boundary or edge between a first and second surface of the object;
ii. A visual boundary wherein a continuous surface of the object disappears from vision;
iii. A continuous marking or plurality of discrete markings on the surface or label of the object.

The object may vary. The object may be an imaging device. For example, the object may be a thermal scanner, retinal scanner, passive infrared sensor (PIR), or any other medical imaging device such as an x-ray, computed tomography (CT) scan, magnetic resonance imaging (MRI) scan or ultrasound. The imaging device may also be used for facial recognition.

Alternatively, or in addition, the object may be an ophthalmic fluid dispenser, such as an eye-drop dispenser.

The second positioning indicator may become less visible as the guide is moved proximal to the eye, in use.

The visual guide may comprise a third positioning indicator. The third positioning indicator may be located proximal to the first and/or second positioning indicator. Alternatively, the third positioning indicator may be located distal to the first and/or second positioning indicator. In some embodiments, the third positioning indicator may be located between the first and second positioning indicator. Providing a third positioning indicator may provide an additional level of accuracy as the user has multiple positioning guides to use. Alternatively, or in addition, the third positioning indicator may enable the user to use two of the three positioning indicators to position the object within a predetermined range of distances relative from the eye and a different combination of two positioning indicators to positioning the object within a predetermined range of angles relative to the visual axis of the user’s eye.

In some embodiments, the visual guide may comprise a plurality of positioning indicators. For example, the visual guide may comprise as 2, 3, 4, 5, 6, 7, 8 or more than 8 positioning indicators.

The invention will now be further and more particularly described, by way of example only, and with reference to the accompanying drawings.

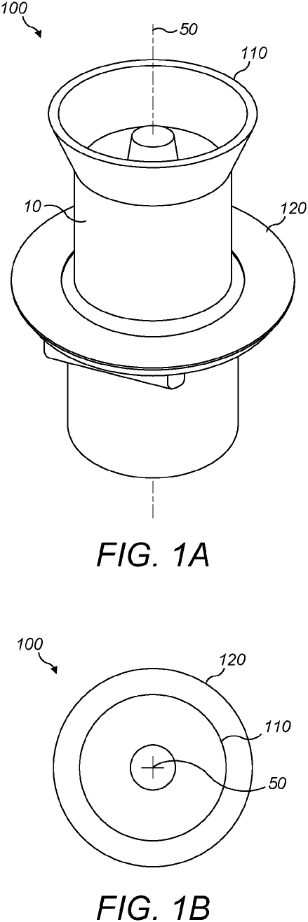

FIG. 1A shows an embodiment of the visual guide.

FIG. 1B shows the visual guide, as shown in FIG. 1A, when viewed by a user, in use.

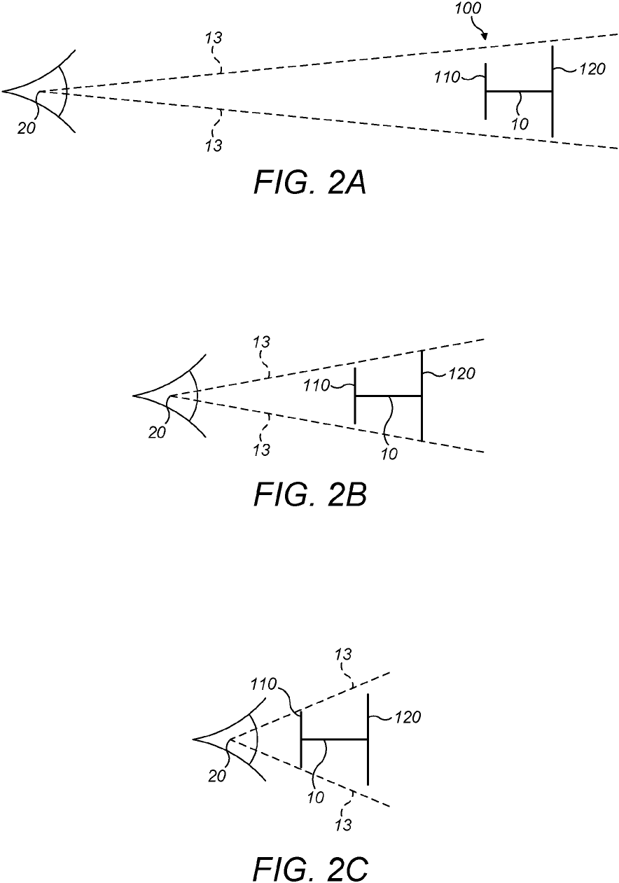

FIG. 2A shows a schematic visual guide comprising a first and second positioning indicator, in use, wherein the object is too far away from the eye.

FIG. 2B shows the visual guide of FIG. 2A, in use, wherein the object is located within a predetermined locus of the user’s eye.

FIG. 2C shows the visual guide of FIG. 2A, in use, wherein the object is too close to the eye.

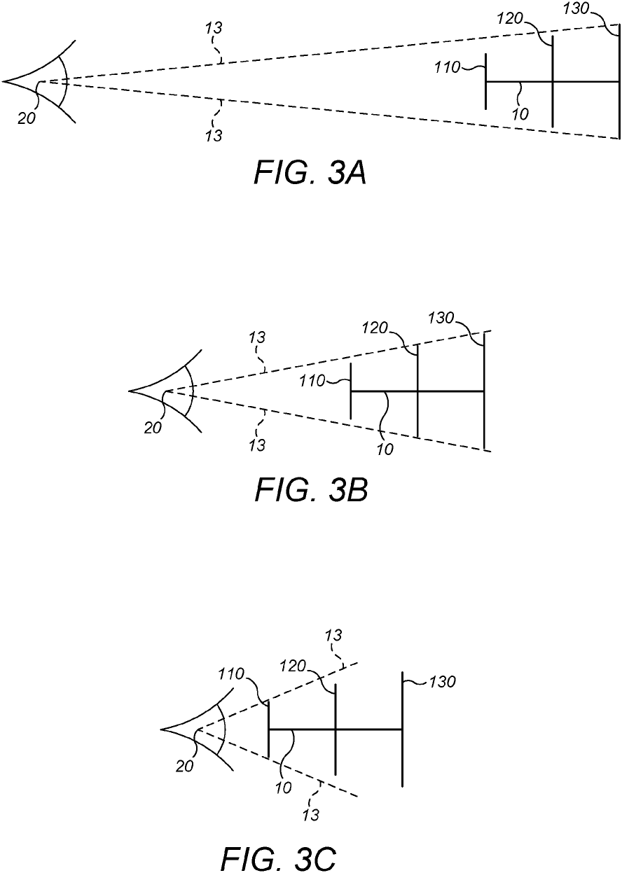

FIG. 3A shows a schematic visual guide comprising a first, second and third positioning indicator, in use, wherein the object is too far away from the eye.

FIG. 3B shows the visual guide of FIG. 3A, in use, wherein the object is located within a predetermined locus of the user’s eye.

FIG. 3C shows the visual guide of FIG. 3A, in use, wherein the object is too close to the eye.

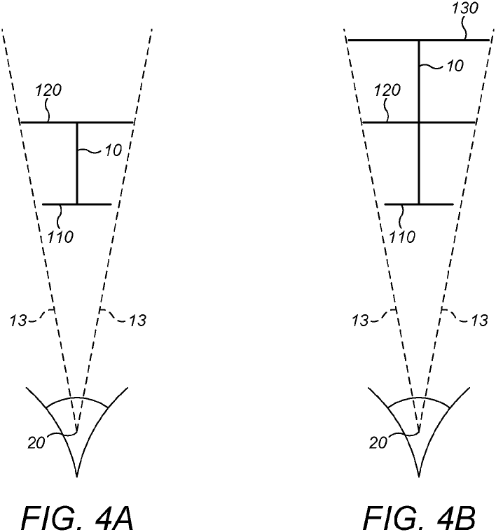

FIG. 4A shows a visual guide comprising a first and second positioning indicator, in use, wherein the visual axis of the user’s eye is substantially vertical.

FIG. 4B shows a visual guide comprising a first, second and third positioning indicator, in use, wherein the visual axis of the user’s eye is substantially vertical.

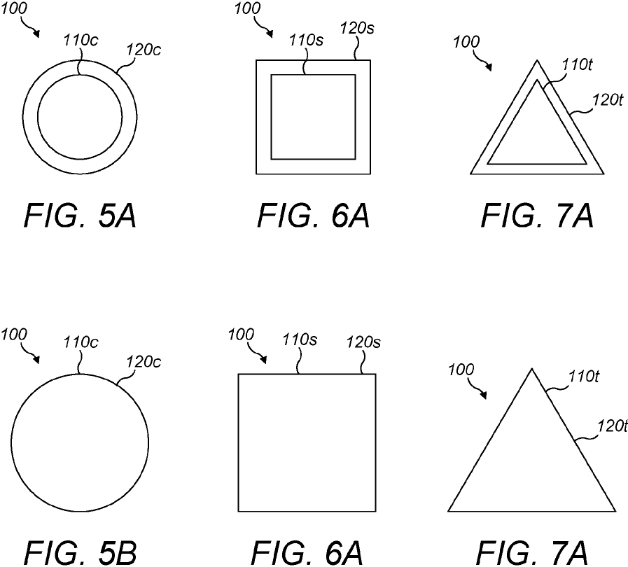

FIG. 5A shows a visual guide as viewed by a user, in use, wherein the object is located outside of the predetermined locus of the user’s eye.

FIG. 5B shows the visual guide of FIG. 5A as viewed by a user, in use, wherein the object is located within the predetermined locus of the user’s eye.

FIG. 6A shows a visual guide as viewed by a user, in use, wherein the object is located outside of the predetermined locus of the user’s eye.

FIG. 6B shows the visual guide of FIG. 6A as viewed by a user, in use, wherein the object is located within the predetermined locus of the user’s eye.

FIG. 7A shows a visual guide as viewed by a user, in use, wherein the object is located outside of the predetermined locus of the user’s eye.

FIG. 7B shows the visual guide of FIG. 7A as viewed by a user, in use, wherein the object is located within the predetermined locus of the user’s eye.

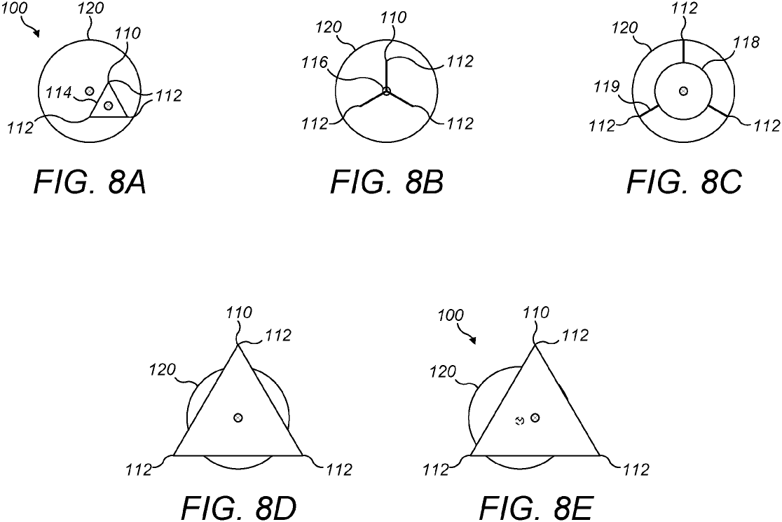

FIG. 8A shows a visual guide as viewed by a user, in use, wherein the object is located outside of the predetermined locus of the user’s eye.

FIG. 8B shows a visual guide as viewed by a user, in use, wherein the object is located outside of the predetermined locus of the user’s eye, but wherein the object is at least approximately aligned with the visual axis of the user’s eye.

FIG. 8C shows a visual guide as viewed by a user, in use, wherein the object is located within the predetermined locus of the user’s eye.

FIG. 8D shows a visual guide as viewed by a user, in use, wherein the object is located outside of the predetermined locus of the user’s eye, but wherein the object is at least approximately aligned with the visual axis of the user’s eye.

FIG. 8E shows a visual guide as viewed by a user, in use, wherein the object is located outside of the predetermined locus of the user’s eye.

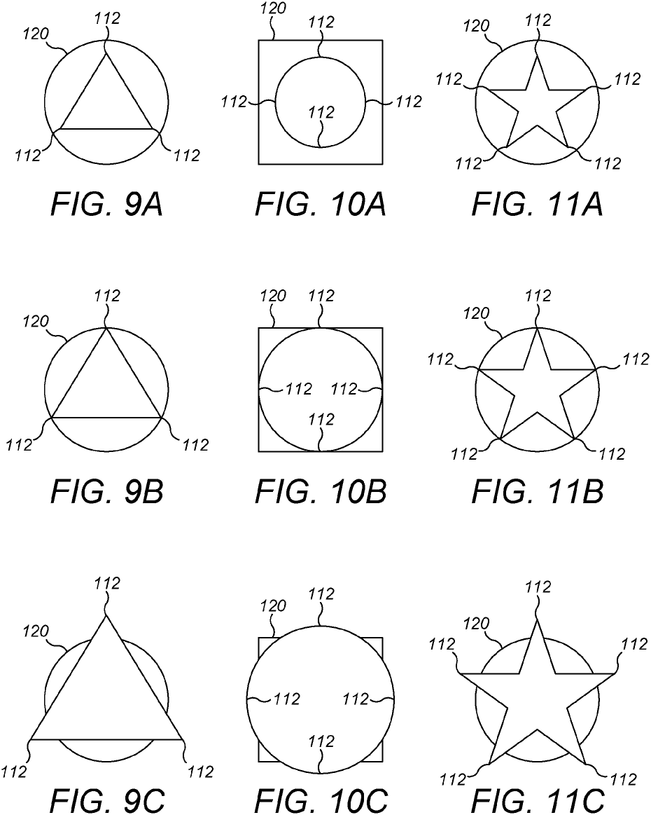

FIG. 9A shows a visual guide, as viewed by a user, wherein the first positioning indicator comprises three discrete elements.

FIG. 9B shows the visual guide of FIG. 9A, as viewed by a user, wherein the object is located within the predetermined locus of the user’s eye.

FIG. 9C shows the visual guide of FIG. 9A, as viewed by a user, wherein the object is located outside of the predetermined locus of the user’s eye.

FIG. 10A shows a visual guide, as viewed by a user, wherein the first positioning indicator comprises four discrete elements.

FIG. 10B shows the visual guide of FIG. 10A, as viewed by a user, wherein the object is located within the predetermined locus of the user’s eye.

FIG. 10C shows the visual guide of FIG. 10A, as viewed by a user, wherein the object is located outside of the predetermined locus of the user’s eye.

FIG. 11A shows a visual guide, as viewed by a user, wherein the first positioning indicator comprises five discrete elements.

FIG. 11B shows the visual guide of FIG. 11A, as viewed by a user, wherein the object is located within the predetermined locus of the user’s eye.

FIG. 11C shows the visual guide of FIG. 11A, as viewed by a user, wherein the object is located outside of the predetermined locus of the user’s eye.

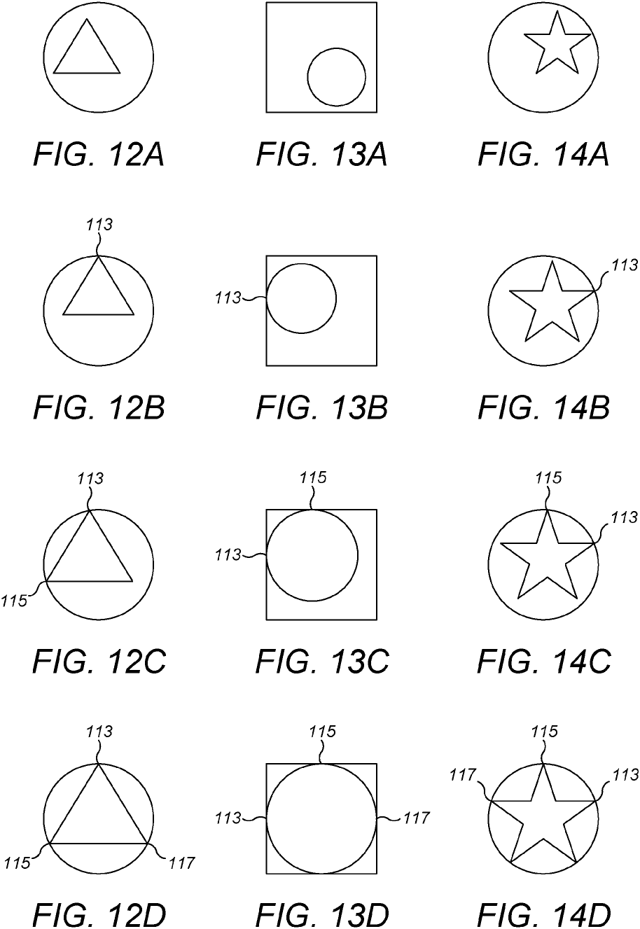

FIG. 12A shows a visual guide, as viewed by a user, wherein there is no visual contact between the first and second positioning indicators.

FIG. 12B shows the visual guide of FIG. 12A, wherein a first portion or discrete element of the first positioning indicator is in visual contact with the second positioning indicator.

FIG. 12C shows the visual guide of FIG. 12A, wherein a first and second portion or discrete element of the first positioning indicator is in visual contact with the second positioning indicator.

FIG. 12D shows the visual guide of FIG. 12A, wherein at least a first, second and third portion or discrete element of the first positioning indicator is in visual contact with the second positioning indicator.

FIG. 13A shows a visual guide, as viewed by a user, wherein there is no visual contact between the first and second positioning indicators.

FIG. 13B shows the visual guide of FIG. 13A, wherein a first portion or discrete element of the first positioning indicator is in visual contact with the second positioning indicator.

FIG. 13C shows the visual guide of FIG. 13A, wherein a first and second portion or discrete element of the first positioning indicator is in visual contact with the second positioning indicator.

FIG. 13D shows the visual guide of FIG. 13A, wherein at least a first, second and third portion or discrete element of the first positioning indicator is in visual contact with the second positioning indicator.

FIG. 14A shows a visual guide, as viewed by a user, wherein there is no visual contact between the first and second positioning indicators.

FIG. 14B shows the visual guide of FIG. 14A, wherein a first portion or discrete element of the first positioning indicator is in visual contact with the second positioning indicator.

FIG. 14C shows the visual guide of FIG. 14A, wherein a first and second portion or discrete element of the first positioning indicator is in visual contact with the second positioning indicator.

FIG. 14D shows the visual guide of FIG. 14A, wherein at least a first, second and third portion or discrete element of the first positioning indicator is in visual contact with the second positioning indicator.

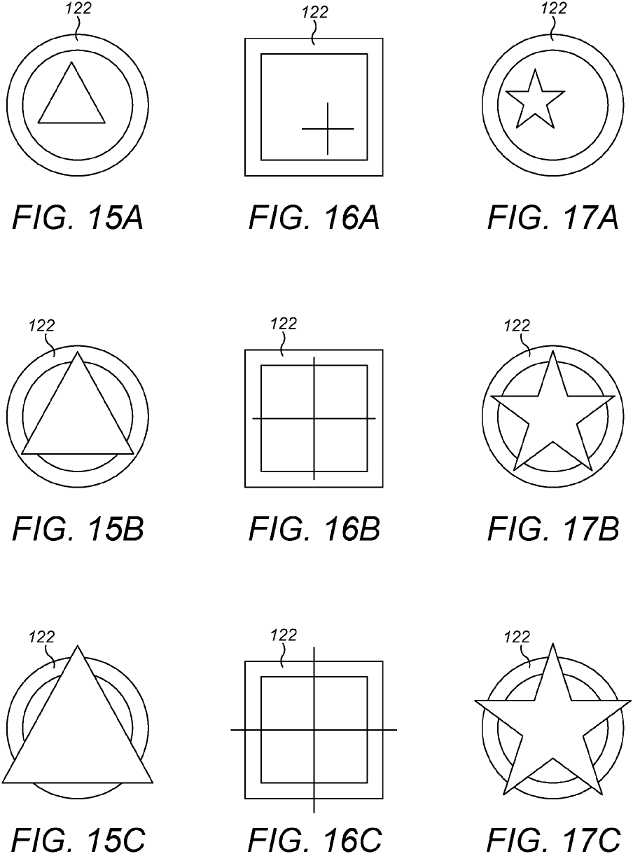

FIG. 15A shows a visual guide wherein the second positioning indicator is defined by an area and wherein the object is located outside of the predetermined locus of the user’s eye.

FIG. 15B shows a visual guide wherein the second positioning indicator is defined by an area and wherein the object is located within the predetermined locus of the user’s eye.

FIG. 15C shows a visual guide wherein the second positioning indicator is defined by an area and wherein the object is located outside of the predetermined locus of the user’s eye.

FIG. 16A shows a visual guide wherein the second positioning indicator is defined by an area and wherein the object is located outside of the predetermined locus of the user’s eye.

FIG. 16B shows a visual guide wherein the second positioning indicator is defined by an area and wherein the object is located within the predetermined locus of the user’s eye.

FIG. 16C shows a visual guide wherein the second positioning indicator is defined by an area and wherein the object is located outside of the predetermined locus of the user’s eye.

FIG. 17A shows a visual guide wherein the second positioning indicator is defined by an area and wherein the object is located outside of the predetermined locus of the user’s eye.

FIG. 17B shows a visual guide wherein the second positioning indicator is defined by an area and wherein the object is located within the predetermined locus of the user’s eye.

FIG. 17C shows a visual guide wherein the second positioning indicator is defined by an area and wherein the object is located outside of the predetermined locus of the user’s eye.

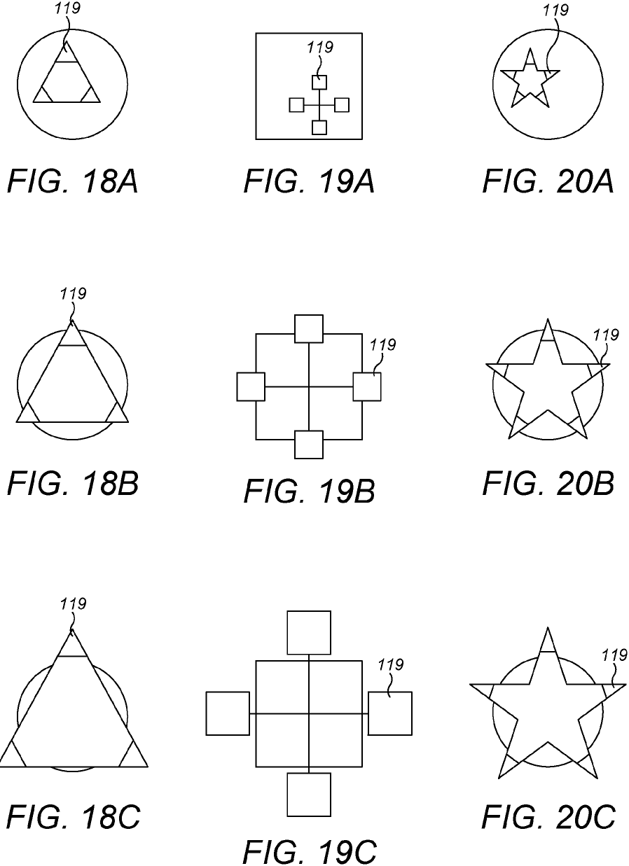

FIG. 18A shows a visual guide wherein the first positioning indicator is defined by an area and wherein the object is located outside of the predetermined locus of the user’s eye.

FIG. 18B shows a visual guide wherein the first positioning indicator is defined by an area and wherein the object is located within the predetermined locus of the user’s eye.

FIG. 18C shows a visual guide wherein the first positioning indicator is defined by an area and wherein the object is located outside of the predetermined locus of the user’s eye.

FIG. 19A shows a visual guide wherein the first positioning indicator is defined by an area and wherein the object is located outside of the predetermined locus of the user’s eye.

FIG. 19B shows a visual guide wherein the first positioning indicator is defined by an area and wherein the object is located within the predetermined locus of the user’s eye.

FIG. 19C shows a visual guide wherein the first positioning indicator is defined by an area and wherein the object is located outside of the predetermined locus of the user’s eye.

FIG. 20A shows a visual guide wherein the first positioning indicator is defined by an area and wherein the object is located outside of the predetermined locus of the user’s eye.

FIG. 20B shows a visual guide wherein the first positioning indicator is defined by an area and wherein the object is located within the predetermined locus of the user’s eye.

FIG. 20C shows a visual guide wherein the first positioning indicator is defined by an area and wherein the object is located outside of the predetermined locus of the user’s eye.

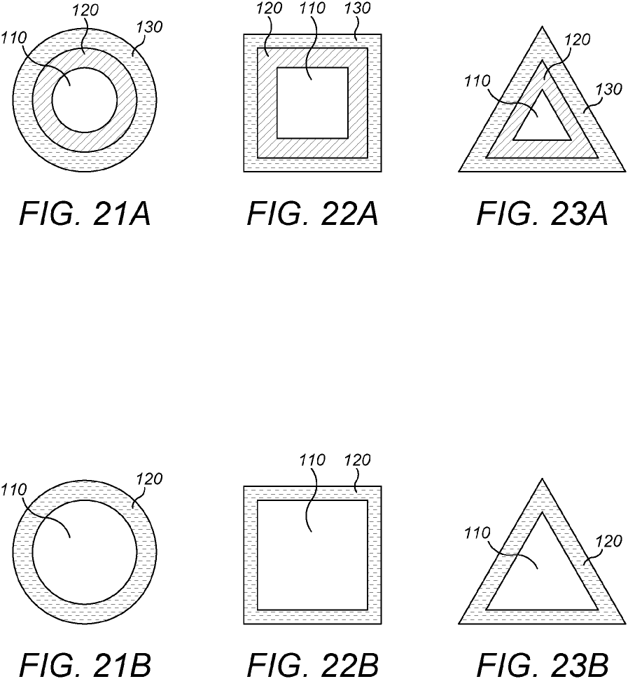

FIG. 21A shows an embodiment of the invention, as viewed by a user, comprising a first, second and third positioning indicator.

FIG. 21B shows an embodiment of the invention, as viewed by a user, comprising a first and second visual guide.

FIG. 22A shows an embodiment of the invention, as viewed by a user, comprising a first, second and third visual guide.

FIG. 22B shows an embodiment of the invention, as viewed by a user, comprising a first and second visual guide.

FIG. 23A shows an embodiment of the invention, as viewed by a user, comprising a first, second and third visual guide.

FIG. 23B shows an embodiment of the invention, as viewed by a user, comprising a first and second visual guide.

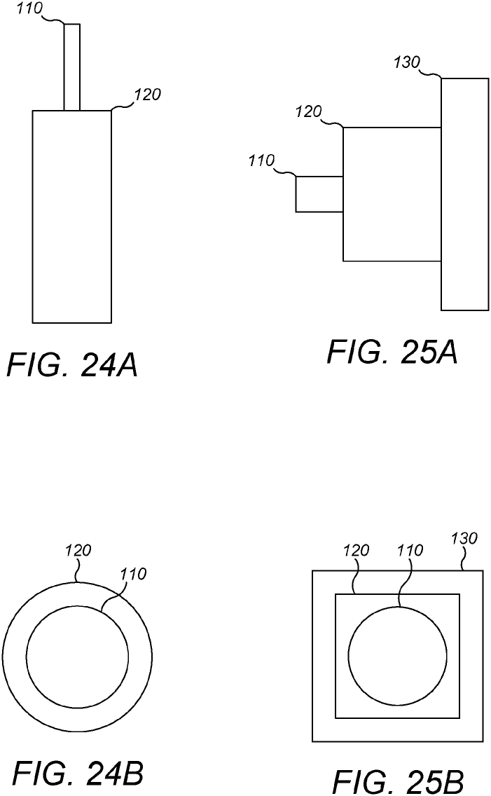

FIG. 24A shows a visual guide wherein the positioning indicators are integral to a fluid dispenser.

FIG. 24B shows the visual guide as shown in FIG. 24A, as viewed by a user, wherein the fluid dispenser is too far away from the user’s eye.

FIG. 25A shows a visual guide, wherein the positioning indicators are integral to an imaging device.

FIG. 25B shows the visual guide as shown in FIG. 25A, as viewed by a user, wherein the imaging device is too far away from the user’s eye.

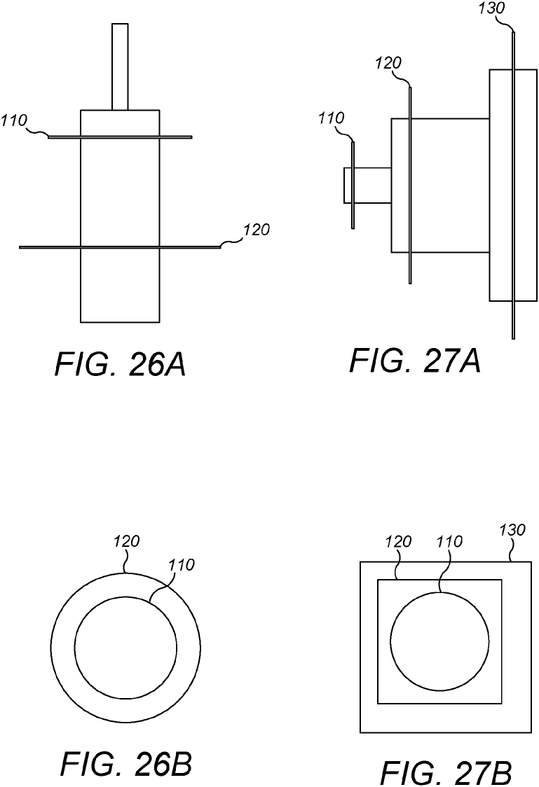

FIG. 26A shows a visual guide, wherein the positioning indicators are attached to a fluid dispenser.

FIG. 26B shows the visual guide as shown in FIG. 26A, as viewed by a user, wherein the fluid dispenser is too far away from the eye.

FIG. 27A shows a visual guide, wherein the positioning indicators are attached to an imaging device.

FIG. 27B shows the visual guide as shown in FIG. 21A, as viewed by a user, wherein the imaging device is too far away from the eye.

FIG. 1A shows a visual guide 100 for assisting a user with the positioning of an object 10 relative to the user’s eye. The object 10 may be any object, such as, but not limited to, an imaging device or an ophthalmic fluid dispenser. The visual guide 100 comprises a first positioning indicator 110 and a second positioning indicator 120, wherein the first positioning indicator 110 is offset from the second positioning indicator 120 along an axis 50.

FIG. 1B shows the visual guide 100, as shown in FIG. 1A, as viewed by a user, in use.

FIGS. 2A-C each show a schematic visual guide 100 at a plurality of distances from the user’s eye 20, wherein the visual guide comprises a first positioning indicator 110 and a second positioning indicator 120. In FIG. 2A, the object 10 is too far away from the eye 20 and therefore the object is outside of the predetermined locus of the user’s eye. Consequently, the user will be able to see both the first positioning indicator 110 and the second positioning indicator 120, as indicated by the dashed lines 13, which represent the boundary of user’s field of view. The first and second indicators will not be in visual contact. As the user moves the object towards the eye, the visual guide will transition into the configuration shown in FIG. 2B. In FIG. 2B, the object is located within the predetermined locus of the user’s eye and thus the first positioning indicator 110 will become in visual contact with the second positioning indicator 120. This is indicated by the dashed lines 13, which show that the positioning indicators 110 120 are both located at the boundary of the user’s field of view. In FIG. 2B, the object is optimally positioned with respect to the user’s eye. As the user continues to move the object close to their eye, the visual guide will transition into the configuration shown in FIG. 2C. In FIG. 2C, the object 10 is too close to the eye 20 such that the object is outside of the predetermined locus of the user’s eye. Once again, the first and second indicators will not be in visual contact. In FIG. 2C, the first positioning indicator 110 may completely obscure the view of the second positioning indicator 120, as indicated by the dashed lines 13.

FIGS. 3A to C each show a schematic visual guide 100 at a plurality of distances from the user’s eye 20, wherein the visual guide comprises a first positioning indicator 110, a second positioning indicator 120 and a third positioning indicator 130. In FIG. 3A, the object 10 is too far away from the eye 20 such that the object is outside of the predetermined locus of the user’s eye. Consequently, the user will be able to see the first, second and third positioning indicators 110, 120, 130, as indicated by the dashed lines 13, which represent the boundary of user’s field of view. The positioning indicators will not be in visual contact. As the user moves the object towards the eye, the visual guide will transition into the configuration shown in FIG. 3B. In FIG. 3B, the object is located within the predetermined locus of the user’s eye, wherein the first, second and third positioning indicators will become in visual contact, as indicated by the dashed lines 13. In FIG. 3B, the object is optimally positioned with respect to the user’s eye. As the user continues to move the object close to their eye, the visual guide will transition into the configuration shown in FIG. 3C. In FIG. 3C, the object 10 is too close to the eye 20 such that the object is outside of the predetermined locus of the user’s eye. Once again, the first, second and third indicators will not be in visual contact. In FIG. 3C, the first positioning indicator 110 may completely obscure the view of the second positioning indicator 120 and the third positioning indicator 130, as indicated by the dashed lines 13.

In some embodiments, not shown, the visual guide comprises more than three positioning indicators, such as 4, 5, 6, 7, 8, 9, 10 or more than 10 positioning indicators. It will be appreciated that any number of positioning indicators may be used.

FIGS. 4A and 4B each show a schematic of a visual guide for assisting a user with positioning an object within a predetermined locus of the user’s eye, wherein the visual axis of the user’s eye is substantially vertical. In this situation, the object may be positioned directly above the eye. However, it will be appreciated that the visual guide can be used to position an object within a predetermined locus of a user’s eye when the visual axis of the user’s eye is at any angle relative to a substantially horizontal axis. For example, in FIGS. 4A and 4B, the visual axis of the user’s eye is substantially perpendicular to a substantially horizontal axis, whereas in FIGS. 2A to 2C and 3A to 3C the visual axis of the user’s eye is substantially parallel to a substantially horizontal axis. In use, the visual axis of the user’s eye may be less than 10, 20, 30, 40, 50, 60, 70, 80 or 90 degrees from a substantially horizontal axis.

FIGS. 5A, 6A, and 7A each show a visual guide 100, as viewed by a user, in use. They each comprise a first positioning indicator 110 and a second positioning indicator 120. In each of these Figures, the first positioning indicator 110 is defined by the boundary or perimeter of a shape, such as a circle 110c, square 110s or triangle 110t. However, it will be appreciated that any shape may be used. The second visual indicator is also defined by the boundary or perimeter of a shape, such as a circle 120c, square 120s or triangle 120t. Again, it will be appreciated that any shape may be used. In each of FIGS. 5A, 6A, and 7A, the first and second positioning indicators are not in visual contact, thus the object is located outside of the predetermined locus of the user’s eye. More specifically, the first positioning indicator 110 is entirely within the boundary of the second positioning indicator 120, therefore, the object is too far away from the user’s eye.

That being said, in FIGS. 5A, 6A and 7A, the first positioning indicator 110 appears to be centralized and/or concentric with the second positioning indicator 120. Consequently, the object is, at least approximately, axially aligned with the visual axis of the user’s eye.

FIGS. 5B, 6B, and 7B each show the visual guide 100 of FIGS. 5A, 6A, and 7A, respectively, as viewed by a user, in use. However, in each of the FIGS. 5B, 6B, and 7B, the first and second positioning indicators are now in visual contact, thus the object is located within the predetermined locus of the user’s eye. More specifically, substantially all of the first positioning indicator 100 is in visual contact with substantially all of the second positioning indicator 120.

FIGS. 8A to 8E each show a visual guide 100, as viewed by a user, in use, wherein the visual guide comprises a first positioning indicator 110 and a second positioning indicator 120. In FIGS. 8A-E, the second positioning indicator 120 is the boundary or perimeter of a shape. More specifically the second positioning indicator 120 is the entire boundary or perimeter of a circle, which has been kept consistent in FIGS. 8A-E for clarity. It will be appreciated that the second positioning indicator may take many forms, shapes, and sizes.

In FIGS. 8A to E, the first positioning indicator 110 comprises a plurality of discrete elements 112. The plurality of discrete elements 112 makes up the entire first positioning indicator 110. In FIG. 8A, each tip of a triangle is a discrete element 112. Alternatively, or in addition, each discrete element is a portion of the first positioning indicator. Consequently, the first positioning indicator 110 comprises a plurality of discrete elements 112 that are connected to form a shape 114, such as a triangle. Alternatively, the first positioning indicator 110 is a shape comprising a plurality of portions, wherein each portion may be a discrete element 112.

In some embodiments, not shown, the second positioning indicator comprises a plurality of discrete elements. More specifically, the plurality of discrete elements 112 makes up the entire second positioning indicator 110. Alternatively, or in addition, each discrete element may be a portion of the second positioning indicator.

Any shape and/or any number of discrete elements may be used. For example, in some embodiments, not shown, the first and/or second positioning indicators comprise 2, 3, 4, 5, 6, 7, 8 or more than 8 discrete elements.

In FIG. 8B, the plurality of discrete elements 112 is connected at a single point 116. Alternatively, in FIG. 8C, the plurality of discrete elements 112 each connect to a shape 118 via a plurality of connection members 119. In some embodiments, the discrete elements 112 comprise the connection members 119. Alternatively, or in addition, the discrete elements 112 may be the connection members 119.

Returning to FIG. 8A, the first positioning indicator 110, or the plurality of discrete elements 112 thereof, is not in visual contact with the second positioning indicator, thus the object is not located within the predetermined locus of the user’s eye. More specifically, the first positioning indicator is contained entirely within the boundary of the second positioning indicator and thus the object is too far away from the user’s eye. Moreover, it appears that the first positioning indicator is not centralised, or concentric, to the second positioning indicator, thus the object is not axially aligned with the visual axis of the user’s eye.

In FIG. 8B, the first positioning indicator is not in visual contact with the second positioning indicator, thus the object is again not located within the predetermined locus of the user’s eye. However, in FIG. 8B the first positioning indicator appears to be, at least approximately, centralised, or concentric, with the second positioning indicator, thus the object is, at least approximately, axially aligned with the visual axis of the user’s eye.

It will however be appreciated that the accuracy with which the alignment of the object with the visual axis of the user’s eye can be determined is somewhat limited when the object is located outside of the predetermined locus of the user’s eye. Nevertheless, as the object moves closer to the predetermined locus of the user’s eye, the accuracy with which the object can be aligned with the visual axis of the user’s eye increases.

FIG. 8C shows a visual guide, as viewed by the user, wherein substantially all of the first positioning indicator 110 is in visual contact with the second positioning indicator 120. More specifically, each of the plurality of discrete elements 112 of the first positioning indicator 110 is in visual contact with the second positioning indicator 120. Consequently, the object is positioned within the predetermined locus of the user’s eye.

FIG. 8D shows a visual guide, as viewed by a user, wherein the first positioning indicator is not in visual contact with the second positioning indicator 120. More specifically, the plurality of discrete elements 112 is not in visual contact with the second positioning indicator 120. The object is therefore located outside of the predetermined locus of the user’s eye. The first positioning indicator is entirely outside of the boundary of the second positioning indicator and therefore the object is too close to the eye.

In FIG. 8D, the first positioning indicator 110 and/or the plurality of discrete elements thereof 112 appears to be, at least approximately, centralized, or concentric, with the second positioning indicator 120, thus the object is, at least approximately, axially aligned with the visual axis of the user’s eye. However, it will again be appreciated that the accuracy with which the alignment of the object with the visual axis of the user’s eye is somewhat limited when the object is outside of the predetermined locus of the user’s eye. Nevertheless, as the object moves closer to the predetermined locus of the user’s eye, the accuracy with which the object can be aligned with the visual axis of the user’s eye increases.

FIG. 8E shows a visual guide, as viewed by a user, wherein the first positioning indicator and/or the plurality of discrete elements 112 are not in visual contact with the second positioning indicator 120. It can therefore be determined that the object is outside of the predetermined locus of the user’s eye. More specifically, the first positioning indicator is entirely outside of the boundary of the second positioning indicator and therefore the object is too close to the eye. In FIG. 8E, the first positioning indicator also appears to be off-centre and therefore not concentric with the second positioning indicator. Consequently, the object is both too close and not correctly aligned with the visual axis of the user’s eye.

FIGS. 9A, 10A and 11A each show a visual guide, as viewed by a user, wherein the object is too far away from the eye. In FIG. 9A, the first positioning indicator comprises three discrete elements 112. In FIG. 10A, the first positioning indicator comprises four discrete elements 112. In FIG. 11A, the first positioning indicator comprises five discrete elements 112.

FIGS. 9B, 10B and 11B each show the visual guides of FIGS. 9A, 10A and 11A, respectively, wherein each of the discrete elements of the first positioning indicator is in visual contact with the second positioning indicator 120, thus the object is located within the predetermined locus of the user’s eye.

FIGS. 9C, 10C and 11C each show the visual guides of FIGS. 9A, 10A and 11A, respectively, wherein each of the discrete elements of the first positioning indicator is not in visual contact with the second positioning indicator. In FIGS. 9C, 10C and 11C the object is too close to the eye and is not located within the predetermined locus of the user’s eye.

FIGS. 12A, 13A and 14A each show a visual guide, as viewed by a user, wherein there is no visual contact between the first and second positioning indicators. Consequently, the object is not located within a predetermined locus of the user’s eye.

FIGS. 12B to D, 13B to D and 14B to D show the visual guides of FIGS. 12A, 13A and 14A, respectively, wherein the object is moving closer to the eye as it passes through figures A to D. More specifically, the object in FIG. 12A is furthest away from the user, 12B is slightly closer, 12C closer still and in FIG. 12D, the object is the closest to the user.

In FIGS. 12B, 13B and 14B, at least a first portion or discrete element 113 of the first positioning indicator 110 is in visual contact with the second positioning indicator 120. Consequently, the object is positioned within a predetermined locus of the user’s eye.

In FIGS. 12C, 13C and 14C, at least a first portion or discrete element 113 and second portion or discrete element 115 of the first positioning indicator 110 is in visual contact with the second positioning indicator 120. Consequently, the object is positioned within a predetermined locus of the user’s eye.

In FIGS. 12D, 13D and 14D, at least a first portion or discrete element 113, a second portion or discrete element 115 and a third portion or discrete element 117 of the first positioning indicator 110 is in visual contact with the second positioning indicator 120. Consequently, the object is positioned within a predetermined locus of the user’s eye.

In some embodiments, it is preferable for at least a first portion or discrete element 113 of the first positioning 110 indicator to be in visual contact with the second positioning indicator 120. Alternatively, or in addition, it may be more preferable for at least a first portion or discrete element 113 and second portion or discrete element 115 of the first positioning 110 indicator to be in visual contact with the second positioning indicator 120. Alternatively, or in addition, it may be most preferable for at least a first portion or discrete element 113, a second portion or discrete element 115 and a third portion discrete element 117 of the first positioning 110 indicator to be in visual contact with the second positioning indicator 120.

In some embodiments, it is preferable for a plurality of portions or discrete elements of the first positioning indicator to visual contact the second positioning indicator. For example, it may be preferable for at least 2, 3, 4, 5, 6, 7, 8 or 9 portions or discrete elements of the first positioning indicator to visual contact the second positioning indicator.

The first positioning indicator may comprise at least 2 portions or discrete elements. In some embodiments, the first positioning indicator comprises at least, 3, 4, 5 or more than 5 portions or discrete elements.

The second positioning indicator may comprise at least 2 portions or discrete elements. In some embodiments, the second positioning indicator comprises at least, 3, 4, 5 or more than 5 portions or discrete elements.

A third positioning indicator may comprise at least 2 portions or discrete elements. In some embodiments, the third positioning indicator comprises at least, 3, 4, 5 or more than 5 portions or discrete elements.

The skilled person will appreciate that the accuracy of a user’s visual perception and fine motor control will limit the accuracy with which the device is positioned with respect to the eye. The positioning indicators will have a thickness which provides a tolerance in the accuracy of the location of the object. As the thickness of the positioning indicators is increased, the volume of the predefined locus will increase, hence increasing the tolerance within which the visual guide may operate.

In some embodiments, each positioning indicator is considered to be an area, wherein the area is defined by the area between two boundaries of a positioning indicator. The area may be varied in order to reflect the accuracy within which the object is required to be located relative to the eye. For example, increasing the distance between two boundaries of at least one of the positioning indicators may increase the size of the predetermined locus. Increasing the distance between two boundaries may be achieved by increasing the thickness of a line, for example.

FIGS. 15A, 16A and 17A each show a visual guide comprising a first positioning indicator and a second positioning indicator. The second positioning indicator is defined by an area 122. The area 122 may be defined by the thickness of a line, perimeter or boundary. Alternatively, the area 122 may be defined by an area between two lines or boundaries. This provides a plurality of possible suitable locations for the object within the predetermined locus. In some embodiments, the second positioning indicator may be an area between two shapes. The area provides a larger target in which the first positioning indicator, or at least one discrete element thereof, is able to visually contact the second positioning indicator.

FIGS. 15B, 16B and 17B show the visual guides of FIGS. 15A, 16A and 17A, respectively, wherein the first positioning indicator, or at least one discrete element thereof, is in visual contact with the second positioning indicator, thus the object is located within the predetermined locus of the user’s eye.

FIGS. 15C, 16C and 17C show the visual guides of FIGS. 15A, 16A and 17A, respectively, wherein the first positioning indicator is not in visual contact with the second positioning indicator, thus the object is located outside of the predetermined locus of the user’s eye.

FIGS. 18A, 19A and 20A each show a visual guide comprising a first positioning indicator and a second positioning indicator. The first positioning indicator, or at least one discrete element thereof, is defined by an area 119. The area 119 may be defined by the thickness of a line, perimeter or boundary. Alternatively, the area 119 may be defined by an area between two lines or boundaries or the area within one boundary, such as the perimeter of a shape. This provides a plurality of possible suitable locations for the object within the predetermined locus. The area provides a larger target in which the first positioning indicator, or at least one discrete element thereof, is able to visually contact the second positioning indicator.

FIGS. 18B, 19B and 20B show the visual guides of FIGS. 18A, 19A and 20A, respectively, wherein the first positioning indicator, or at least one discrete element thereof, 119 is in visual contact with the second positioning indicator, thus the object is located within the predetermined locus of the user’s eye.

FIGS. 18C, 19C and 20C show the visual guides of FIGS. 18A, 19A and 20A, respectively, wherein the first positioning indicator is not in visual contact with the second positioning indicator 122, thus the object is located outside of the predetermined locus of the user’s eye.

FIGS. 21A, 22A and 23A show a visual guide comprising a plurality of positioning indicators. More specifically, the visual guide comprises a first 110 second 120 and third 130 positioning indicator, wherein each of the positioning indicators, or at least one of the discrete elements thereof, comprise a pattern and/or colour. Moreover, each of the patterns and/or colours is visually different.

FIGS. 21B, 22B and 23B show a visual guide comprising a first 110 and second 120 positioning indicator, wherein each of the positioning indicators, or at least one of the discrete elements thereof, comprise a pattern and/or colour. Moreover, each of the patterns and/or colours is visually different.

FIGS. 24A and 25A each show a visual guide, wherein the positioning indicators are integral to the object. FIG. 24A shows a fluid dispenser, such as an ophthalmic fluid dispenser, wherein a first surface, such as the dispensing tip or nozzle, is a first positioning indicator 110 and a second surface, such as the shoulder or main body of the bottle, is a second positioning indicator. In some embodiments, not shown, the fluid dispenser may comprise more than two positioning indicators. FIG. 24B shows the visual guide as shown in FIG. 24A, as viewed by a user.

FIG. 25A shows an imaging device, wherein a first surface of the body is a first positioning indicator 110, a second surface of the body is a second positioning indicator 120 and a third surface of the body is a third positioning indicator 130. In some embodiments, not shown, the imaging device may comprise two or more positioning indicators. FIG. 25B shows the visual guide as shown in FIG. 25A, as viewed by a user.

FIGS. 26A and 27A each show a visual guide, wherein the positioning indicators are attached to the object. Alternatively, the positioning indicators may be mounted onto the object. FIG. 26A shows a fluid dispenser, such as an ophthalmic fluid dispenser, wherein a first positioning indicator 110 is attached to a first surface of the dispenser, such as the nozzle or tip. A second positioning indicator is attached to a second surface, such as the shoulder or main body of the dispenser. In some embodiments, not shown, the fluid dispenser may comprise more than 2 positioning indicators. FIG. 26B shows the visual guide as shown in FIG. 26A, as viewed by a user, in use.

FIG. 27A shows an imaging device, wherein a first positioning indicator 110 is attached to a first surface of the device body, a second positioning indicator 120 is attached to a second surface of the device body and a third positioning indicator 130 is attached to a third surface of the device body. In some embodiments, not shown, the imaging device may comprise 2 or more positioning indicators. FIG. 227B shows the visual guide as shown in FIG. 27A, as viewed by a user, in use.

1-16. (canceled)

17. A visual guide for assisting a user with the positioning of an object relative to the user’s eye, the visual guide comprising:
a first positioning indicator provided on an axis; and
a second positioning indicator, visually distinct from the first,
wherein the first and second positioning indicators are offset along the axis and
wherein visual contact of the first and second positioning indicators occurs when the object is located within a predetermined range of distances from the user’s eye and when the object is positioned within a predetermined range of angles relative to the visual axis of the user’s eye.

18. The visual guide according to claim 17, wherein visual contact of substantially all of the first indicator with at least a portion of the second positioning indicator occurs when the object is located within a predetermined range of distances from the eye and when the object is positioned within a predetermined range of angles relative to the visual axis of the user’s eye.

19. The visual guide according to claim 17, wherein the first and second positioning indicators are coaxial.

20. The visual guide according to claim 17, wherein at least one of the length, circumference, diameter and cross-sectional area of the first positioning indicator is less than the corresponding measurement of the second positioning indicator.

21. The visual guide according to claim 17, wherein the first positioning indicator can fit substantially within an external boundary of the second positioning indicator when viewed in two-dimensions (2D).

22. The visual guide according to claim 17, wherein the first positioning indicator is a different and/or contrasting colour to the second positioning indicator.

23. The visual guide according to claim 17, wherein at least one of the first and second positioning indicators is configured for attachment to the object.

24. The visual guide according to claim 17, wherein the first and/or second positioning indicator comprises a plurality of discrete elements.

25. The visual guide according to claim 24, wherein at least one of the length, circumference, diameter and cross-sectional area of at least one element of the first positioning indicator is less than the corresponding measurement of the second positioning indicator.

26. The visual guide according to claim 17, wherein at least one of the first and second positioning indicators, or at least one element thereof, is at least one of:
i. An outer surface or boundary of a ring or disc;
ii. A continuous line or marking on a ring or disc;
iii. A plurality of discrete markings on a ring or disc;
iv. An outer surface of at least three protrusions or outcrops;
v. A continuous marking or scale on at least three protrusions or outcrops.

27. The visual guide according to claim 17, wherein at least one of the first and second positioning indicators is integral to the object.

28. The visual guide according to claim 17, wherein at least one of the first and second positioning indicators, or at least one element thereof, is at least one of:
i. A boundary or edge between a first and second surface of the object;
ii. A visual boundary wherein a continuous surface of the object disappears from vision;
iii. A continuous marking or plurality of discrete markings on the surface or label of the object.

29. The visual guide according to claim 17, wherein the object is an imaging device.

30. A visual guide for assisting a user with the positioning of an ophthalmic fluid dispenser relative to the user’s eye, the visual guide comprising:
a first positioning indicator provided on an axis; and
a second positioning indicator, visually distinct from the first,
wherein the first and second positioning indicators are offset along the axis and
wherein visual contact of the first and second positioning indicators occurs when the dispenser is located within a predetermined range of distances from the user’s eye and when the dispenser is positioned within a predetermined range of angles relative to the visual axis of the user’s eye.

31. The visual guide according to claim 30, wherein the second positioning indicator becomes less visible as the guide is moved proximal to the eye.

32. The visual guide according to claim 30, further comprising a third positioning indicator.
